(19) United States
(12) Patent Application Publication
(10) Pub. No.: US 2014/0249095 A1
(43) Pub. Date: Sep. 4, 2014

(54) Method for Inhibiting Spinocerebellar Ataxia

(71) Applicant: National Normal University, Taipei (TW)

(72) Inventors: Guey-Jen LEE-CHEN, Taipei (TW); Chiung-Mei CHEN, Taipei (TW)

(73) Assignee: National Normal University, Taipei (TW)

(21) Appl. No.: 14/193,130

(22) Filed: Feb. 28, 2014

(30) Foreign Application Priority Data
Mar. 4, 2013 (TW) 102107479

(51) Int. Cl. A61K 31/7048; A61K 36/71

(52) U.S. Cl. CPC A61K 31/7048; A61K 36/71
USPC 514/27

(57) ABSTRACT

A method for inhibiting spinocerebellar ataxia is disclosed, which comprises: administering an extract of Paeonia lactiflora to a subject in need; wherein a concentration of the extract of Paeonia lactiflora is in the range from 1 μg/mL to 80 μg/mL.

### CROSS REFERENCE TO RELATED APPLICATION

This application claims the benefits of the Taiwan Patent Application Serial Number 102107479, filed on March 4, 2013, the subject matter of which is incorporated herein by reference.

### BACKGROUND OF THE INVENTION

1. Field of the Invention

The present invention relates to a method for inhibiting spinocerebellar ataxia, and particularly to a method for inhibiting spinocerebellar ataxia relating to suppressing aggregation of polyglutamine with an extract of Paeonia lactiflora.

2. Description of Related Art

Spinocerebellar atrophy is referred to spinocerebellar ataxias (SCAs), which are a complex group of heterogeneous autosomal dominant neurodegenerative disorders characterized by cerebellar dysfunction alone or in combination with other neurological abnormalities.

On the current market, there is no drug for curing or suppressing polyglutamine related spinocerebellar ataxia progression, and the symptom thereof is irreversible: patients may fail to appropriately control their movements at the beginning; with the deterioration of disease condition, patients become failing to walk and write progressively, and finally become failing to talk and swallow. In the worst case, it may bring patients to an end with death. However, even though there is atrophy of the cerebellum, the brainstem, and the spinal cord, the intelligence is completely unaffected, so that patients can be clearly conscious of the fact that their bodies gradually become inactive.

In addition, the surgery, radiation therapy, chemotherapy, hormone therapy, biopharmaceutical therapy, etc. used in Western therapy, usually bring strong side effects to patient's body, thereby causing weakness of patients gradually. Today, traditional Chinese herbal medicine is regarded as a relatively tender way for treatment and generally agreed with people so as to have a very high market acceptance.

In view of the gradually increased global population suffering from spinocerebellar ataxia, if a pharmaceutical composition for inhibiting spinocerebellar ataxia can be found out from a variety of herbal medicines, it is bound to give assistance to the treatment of spinocerebellar ataxia, thereby slowing down disease progression and rendering better quality of life.

### SUMMARY OF THE INVENTION

The above and other objects, features and other advantages of the present invention will be more clearly understood from the following detailed description taken in conjunction with the accompanying drawings. Other objects, advantages, and novel features of the invention will become more apparent from the following detailed description when taken in conjunction with the accompanying drawings.

An object of the present invention is to provide a method for inhibiting spinocerebellar ataxia to give assistance to the treatment of spinocerebellar ataxia, and slow down disease progression.

An object of the present invention is to provide a method for suppressing aggregation of polyglutamine to reduce abnormal aggregation of polyglutamine.

To achieve above object, the present invention provides a method for inhibiting spinocerebellar ataxia, comprising: administering a pharmaceutical composition comprising an extract of Paeonia lactiflora to a subject in need, wherein a concentration of the extract of Paeonia lactiflora is in the range from 1 μg/mL to 80 μg/mL.

The present invention also provides a pharmaceutical composition for inhibiting spinocerebellar ataxia, comprising: an extract of Paeonia lactiflora, wherein a concentration of the extract of Paeonia lactiflora is in the range from 1 μg/mL to 80 μg/mL.

In spinocerebellar ataxia, the expansions of CAG trinucleotide repeats encoding a polyglutamine (polyQ) stretch have been shown to cause dominantly inherited SCA1, SCA2, SCA3, SCA6, SCAT, SCA17 and dentatorubropallidoluy-sianatrophy (DRPLA). These polyQ-mediated genetic disorders in SCAs have shown selective progressive degeneration of the cerebellum, brainstem, and spinal cord tract, with prominent pathological hallmark of intranuclear and cytoplasmic accumulation of aggregated polyQ proteins inside degenerated neurons, thereby causing the dysfunction and degeneration of specific neurons.

Accordingly, the present invention further provides a method for suppressing aggregation of polyglutamine, comprising: administering a pharmaceutical composition comprising an extract of Paeonia lactiflora to a subject in need, wherein a concentration of the extract of Paeonia lactiflora is in the range from 1 μg/mL to 80 μg/mL.

The present invention also provides a pharmaceutical composition for suppressing aggregation of polyglutamine, comprising: an extract of Paeonia lactiflora, wherein a concentration of the extract of Paeonia lactiflora is in the range from 1 μg/mL to 80 μg/mL.

Preferably, the concentration of the extract of Paeonia lactiflora is in the range from 1.5 μg/mL to 55 μg/mL, and the extract of Paeonia lactiflora comprises at least one active component selected from a group consisting of paeoniflorin and albiflorin, but the present invention is not limited thereto.

When the pharmaceutical composition of the present invention includes paeoniflorin and/or albiflorin, the concentration of the paeoniflorin and albiflorin are not particularly limited, and may be adjusted according to actual situation for use. Preferably, the paeoniflorin may have a concentration of 50 nM to 300 nM, and albiflorin may have a concentration of 3 μM to 10 μM. In other word, the effective doses of the paeoniflorin and albiflorin included in the pharmaceutical composition may be changed according to the administering pathway, the used excipient, and the possibility of combination with other pharmaceuticals, and those of ordinary skill in the art can modify the dose required for a subject to obtain expected treatment effect.

According to the requirement for use, the pharmaceutical composition of the present invention may further comprise at least one of a pharmaceutically acceptable carrier, a diluent, or an excipient in the art. For example, the extract of Paeonia lactiflora is encapsulated into liposome to facilitate delivery and absorption; the extract of Paeonia lactiflora is diluted with aqueous suspension, dispersion or solution to facilitate injection; or the extract of Paeonia lactiflora is prepared in a form of a capsule or tablet for storage and carrying. In addition, the pharmaceutical composition of the present invention may also be administered with any conventional drug or additive together, as long as without reducing the treatment effect of the pharmaceutical composition of the present invention.

The pharmaceutical composition of the present invention may be purchased on the market, or may be obtained by heating and extracting Paeonia lactiflora in water and filtering out a residue. For example, water which is in an amount of 10 to 20 times of the weight of the Paeonia lactiflora may be mixed with the Paeonia lactiflora to form a mixture, and the mixture is heated to a temperature of 90° C. to 100° C. for 30 minutes to 1 hour, or the mixture is directly heated to has a volume of ¼ to ½ the original volume thereof, to obtain an extract of Paeonia lactiflora. However, the present invention is not limited thereto, and may use any conventional technique to obtain the extract of Paeonia lactiflora. Furthermore, the extract of Paeonia lactiflora may be formed in a dry form by a drying process, such as spray drying method, freeze-drying method, scientific Chinese herbal medicine granulation method, to be prepared into a health food and a clinical therapeutic pharmaceutical for the treatment and the prevention of spinocerebellar ataxia.

The term “inhibit” refers to the case that the pharmaceutical composition including the extract of Paeonia lactiflora of the present invention is applied to a subject suffering from spinocerebellar ataxia, having symptom of spinocerebellar ataxia, or having a tendency of development of spinocerebellar ataxia, in order to achieve the treatment, mitigation, slowing, therapy, improvement, or recovery of the tendency of the disease and symptoms.

To implement the method according to the present invention, the above pharmaceutical composition can be administered via oral administering, parenteral administering, inhalation spray administering, topical administering, rectal administering, nasal administering, sublingual administering, vaginal administering, or implanted reservoir, and so on. The term “parenteral” used here refers to subcutaneous injection, intradermal injection, intravenous injection, intramuscular injection, intraarticular injection, intraarterial injection, joint fluid injection, intrathoracic injection, intrathecal injection, injection at morbid site, and intracranial injection or injection technique.

### BRIEF DESCRIPTION OF THE DRAWINGS

FIG. 1A shows a Western blot analysis of ATXN3/Q₁₄₋₇₅-GFP protein expression induced by doxycycline in 293 cells according to a preferable example of the present invention.

FIG. 1B shows a real-time PCR quantification of RNA expression in ATXN3/Q₁₄₋₇₅-GFP 293 cell induced by doxycycline according to a preferable example of the present invention.

FIG. 2A shows chromatographic patterns from HPLC analysis (230 nm) of the extract of P. lactiflora (Paeonia lactiflora) according to a preferable example of the present invention.

FIG. 2B shows the cytotoxicity of the extract of P. lactiflora, paeoniflorin, gallic acid, albiflorin and histone deacetylase inhibitor (HDAC inhibitor) SAHA (suberoylanilide suberoylanilide hydroxamic acid) against HEK-293 cells using MTT viability assay according to a preferable example of the present invention.

FIG. 2C shows the cytotoxicity of the extract of P. lactiflora, paeoniflorin, gallic acid, albiflorin and SAHA against SH-SY5Y cells using MTT viability assay according to a preferable example of the present invention.

FIG. 3 shows the aggregation analysis of ATXN3/Q₇₅-GFP cells untreated or treated with extract of P. lactiflora (2˜200 μg/mL), paeoniflorin, gallic acid, albiflorin and SAHA (100 nM˜5 μM) according to a preferable example of the present invention.

FIG. 4A shows Western blot analysis of protein expression in ATXN3/Q₇₅-GFP SH-SY5Y cells induced by doxycycline according to a preferable example of the present invention.

FIG. 4B shows the analysis of aggregation in ATXN3/Q₇₅-GFP SH-SY5Y cells untreated or treated with P. lactiflora (10 μg/mL) or paeoniflorin (100 nM) according to a preferable example of the present invention.

### DETAILED DESCRIPTION OF THE PREFERRED EMBODIMENT

P. lactiflora Extract Preparation and HPLC Analysis

The extract from P. lactiflora used in the following experiments was provided by Sun-Ten Pharmaceutical Company (Taipei, Taiwan). Briefly, 100 g of dried P. lactiflora was boiled with 1500 mL of water at 100° C. for 30 min and was sieved using a 100-mesh sieve. The extract was concentrated to 100 mL and filtered using a 200-mesh sieve. The extract was then dried by speed vacuum concentration and then stored at -20° C. until used.

High performance liquid chromatography (HPLC) analysis was performed using a LaChoursom Elite HPLC system (Hitachi), consisting /f a photo diode array detector. The chromatographic separation of P. lactiflora extract (50 μL, 1 mg/mL) was carried out on a Hypersil ODS (C18) column (250×4.6 mm, 5 μm), eluted with the mixture of 0.1% formic acid in water (A) or acetonitrile (B). The linear gradient elution program for A:B (v/v) was set as follows: 95:5 (0-10 min), 95:5-70:30 (10-40 min), 70:30-15:85 (40-55 min), 15:85-95:5 (55-60 min), 95:5 (60-75 min) with a flow rate of 1 mL/min. Absorbance was monitored at 230, 250, 270 nm and the scan range for photo diode array was 190˜400 nm. Paeoniflorin, gallic acid and albiflorin (21[10 μL, 20 mM) were used as reference compounds for P. lactiflora.

Cell Culture and Cell Proliferation Assay

Human embryonic kidney HEK-293 cells (ATCC No. CRL-1573) were cultivated in Dulbecco's modified Eagle's medium (DMEM) containing 10% fetal bovine serum (FBS). Human neuroblastoma SH-SYSY cells (ATCC No. CRL-2266) were maintained in DMEM F12 supplemented with 10% FBS. Cells were cultivated at 37° C. incubator containing 5% CO₂ and cell proliferation was measured based upon the reduction of the tetrazolium salt, 3,[4,5-dimethylthiazol-2-yl]-2,5-diphenyl-tetrazolium bromide (MTT). Cells were plated into 48-well (5×10⁴/well) dishes, grown for 20 hours and treated with different concentrations of the P. lactiflora extract (5˜30 mg/mL) or pure compound (100 nM˜1 mM). After one day, 20 μL MTT (5 mg/mL in PBS, Sigma) was added to cells and incubated for 2 hours. The absorbance of the purple formazan dye was measured at 570 nm by a Bio-Tek μQuant Universal Microplate Spectrophotometer.

ATXN3 cDNA Constructs

Polyadenylated RNA (200 ng) isolated from neuroblastoma SK-N-SH cells was reverse transcribed using the SuperScript™III reverse transcriptase (Invitrogen). The sense and antisense primers used for ATXN3/Q₁₄ cDNA (+826˜+1152, NM_{—}004993) amplification were 5′-ATTCAGCTAAGTATGCAAGGTAGTTCCA (codon for Met257 underlined, SEQ ID NO: 1) and 5′-CATGCCATGGCATGTTTTTTTCCTTCTGTT (NcoI site underlined, SEQ ID NO: 2). The amplified 3′ polyQ-containing cDNA fragment (translated into amino acids 2571[361) was cloned into pGEM-T Easy (Promega) and sequenced. The ATXN3/Q₁₄ cDNA was excised with EcoRI (in pGEM-T Easy vector) and NcoI and subcloned into pEGFP-N1 (Clontech). Then, DNA fragment containing in-frame ATXN3/Q₁₄-EGFP was excised with HindIII-NotI and subcloned into the pcDNAS/FRT/TO. The ATXN3/Q₇₅ cDNA was made by replacing an 88 by ATXN3/Q₁₄ BsmBI-BsmFI fragment with a 271 by ATXN3/Q₇₅ fragment from the cDNA clone of a SCA3 patient.

Isogenic 293 and SH-SY5Y Cell Lines

Human 293-derived Flp-In™-293 cells (Invitrogen) were cultivated in DMEM containing 10% FBS as described. The cloned pcDNA5/FRT/TO-ATXN3/Q₁₄ and Q₇₅ plasmids were used to generate the isogenic ATXN3/Q_{14˜75} cell lines by targeting insertion into Flp-In™-293 cells. These cell lines were grown in medium containing 5 μg/mL blasticidin and 100 μg/mL hygromycin (InvivoGen). In addition, human SH-SYSY-derived Flp-In host cell line was constructed as described, and the SH-SYSY host cells were used to generate isogenic ATXN3/Q_{14˜75} lines and maintained as described above. ATXN3/Q₇₅ Aggregation Assay

293 ATXN3/Q₇₅-GFP cells were plated into 96-well (2×10⁴/well) dishes, grown for 24 hours and treated with different concentrations of the P. lactiflora extract (2˜200 μg/mL) or suberoylanilide hydroxamic acid (SAHA, Cayman Chemical), paeoniflorin (Sigma), gallic acid and albiflorin (Choursomadex) (100 nM˜5 μM) for 8 hours. Then, doxycycline (10 μg/mL, BD) was added to the medium in each well to induce ATXN3/Q₇₅-GFP expression for 6 days. Oxaliplatin (5 μM, Sigma) was also added to increase aggregate accumulation through inhibition of cell division. Then, cells were stained with Hoechst 33342 (0.1 μg/mL, Sigma) and aggregation percentage was assessed by HCA system, with excitation/emission wavelengths at 482/536 (EGFP).

SH-SYSY ATXN3/Q₇₅-GFP cells were seeded in 6-well (2×10⁵/well) plate, with all trans-retinoic acid (10 μM, Sigma) added at seeding time. At day 2, cells were treated with paeoniflorin (100 nM) or the P. lactiflora extract (10 μg/mL) for 8 hours, and then doxycycline (5 μg/mL) was added to induce ATXN3/Q₇₅-GFP expression. The cells were kept in the medium containing 10 μM trans-retinoic acid, doxycycline and paeoniflorin/P. lactiflora extract for 6 days. After that, cells were stained with Hoechst 33342 (0.1 μg/mL) and aggregation percentage was assessed as described.

Real-Time PCR

Total RNA from 293 ATXN3 lines was extracted using Trizol reagent (Invitrogen). The RNA was DNase (Stratagene) treated, quantified, and reverse-transcribed to cDNA. Real-time quantitative PCR experiments were performed in the ABI PRISM® 7000 Sequence Detection System (Applied Biosystems). Amplification was performed on 100 ng cDNA with gene-specific TaqMan fluorogenic probes Hs00245259_ml for ATXN3 and 4326321E for HPRT1 (endogenous control) (Applied Biosystems). Fold change was calculated using the formula 2^{ΔCt}, ΔC_{T}=C_{T} (control)−C_{T} (target), in which C_{T} indicates cycle threshold.

Western Blot Analysis

Total proteins were prepared using lysis buffer containing 50 mM

Tris-HCl, 150 mM NaCl, 1 mM EDTA, 1 mM EGTA, 0.1% SDS and 0.5% sodium deoxycholate, 1% Triton X-100, and protease inhibitor cocktail (Calbiochem). Proteins (25 μg) were separated on 10% SDS-polyacrylamide gel electrophoresis and blotted onto nitrocellulose membranes by reverse electrophoresis. After blocking, the membrane was probed with GFP (1:500 dilution, Santa Cruz) or GAPDH (1:1000 dilution, MDBio) at 4° C. overnight. Then, the immune complexes were detected by horseradish peroxidase-conjugated goat anti-mouse IgG antibody (1:5000 dilution, Jackson ImmunoResearch) or goat anti-rabbit IgG antibody (1:5000 dilution, GeneTex) and chemiluminescent substrate (Millipore).

Statistical Analysis

For each set of values, data were expressed as the means±standard deviation (SD). Three independent experiments were performed and non-categorical variables were compared using the Student's t-test. All P-values were two-tailed, with values of P<0.05 considered significant.

Results

Construction of 293 Cells Expressing ATXN3/Q₇₅ Aggregates

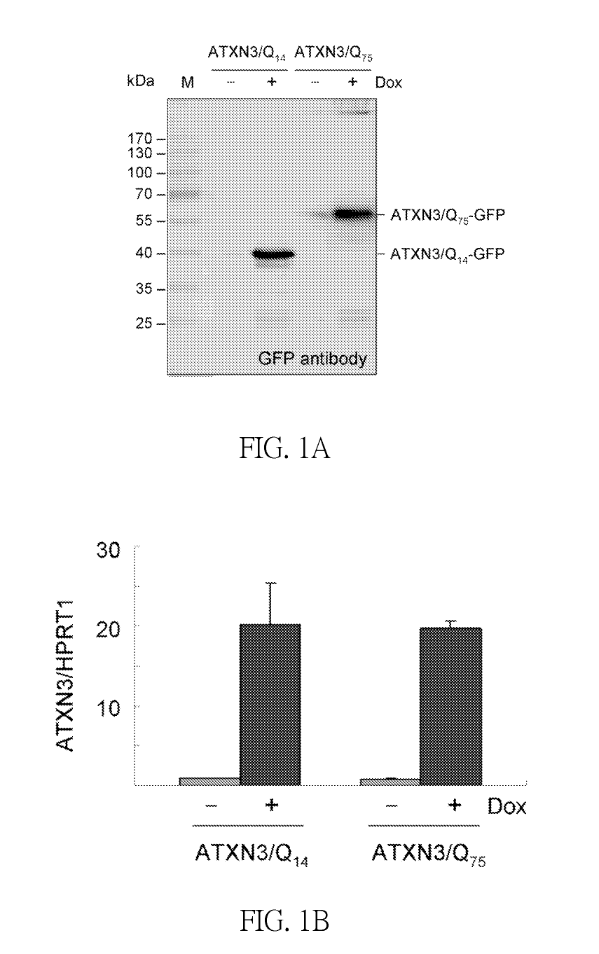

In the present example, GFP-tagged ATXN3 C-terminal Q_{14˜75}-containing fragment was cloned to establish Flp-In 293 cells with ATXN3/Q_{14˜75}-GFP expression in an inducible fashion. As shown in FIG. 1A, the GFP antibody detected 40 kDa ATXN3/Q₁₄-GFP and 57 kDa ATXN3/Q₇₅-GFP proteins in doxycycline (Dox) induced ATXN3 cells. Then, as shown in FIG. 1B, ATXN3-RNA levels were examined by real-time PCR using ATXN3-specific probe and primers, and in the presence of Dox, the two ATXN3 lines expressed about 20 times more ATXN3 RNA than in the absence of Dox. While the expressed ATXN3/Q₁₄ was mainly diffused, the expressed ATXN3/Q₇₅-GFP formed aggregates in the fluorescence microscopy images (not shown).

Extract of P. lactiflora and Constituents

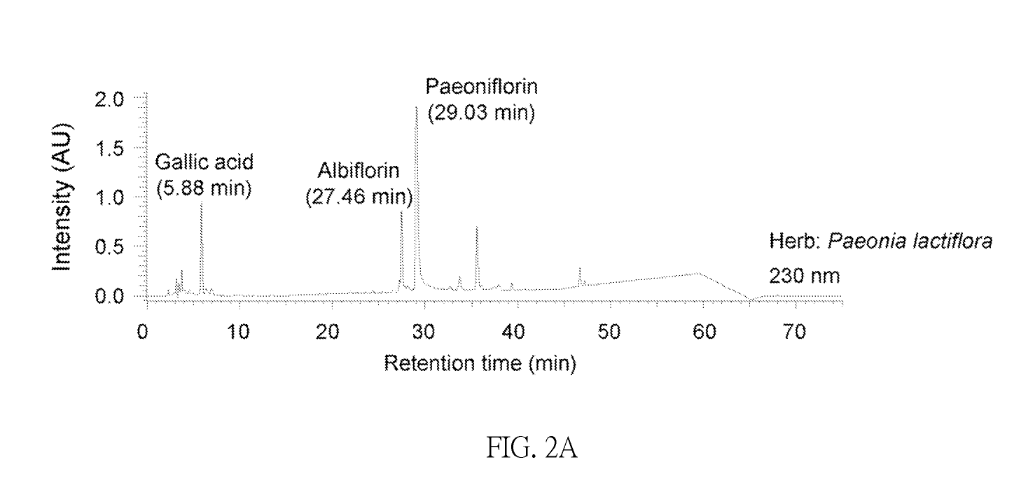

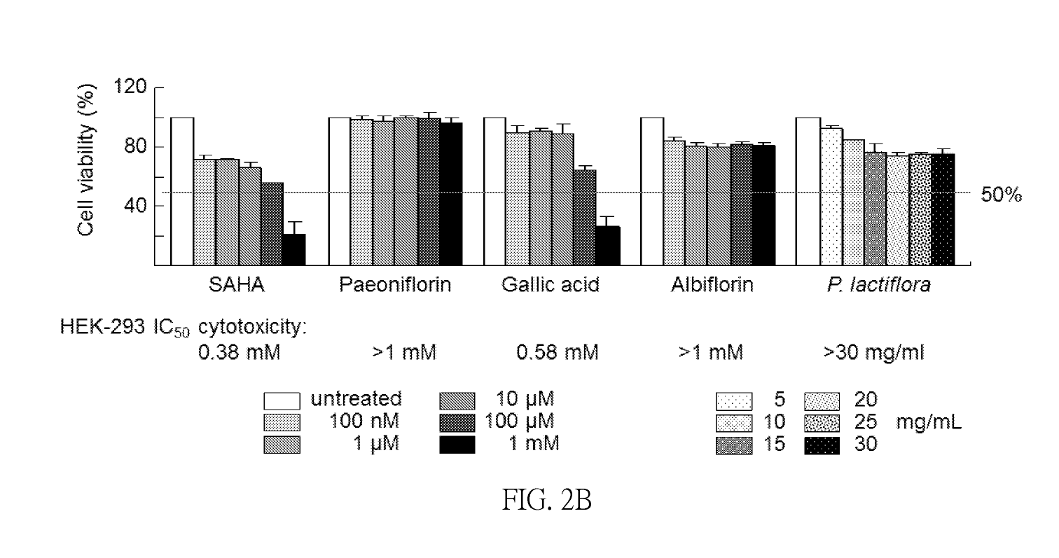

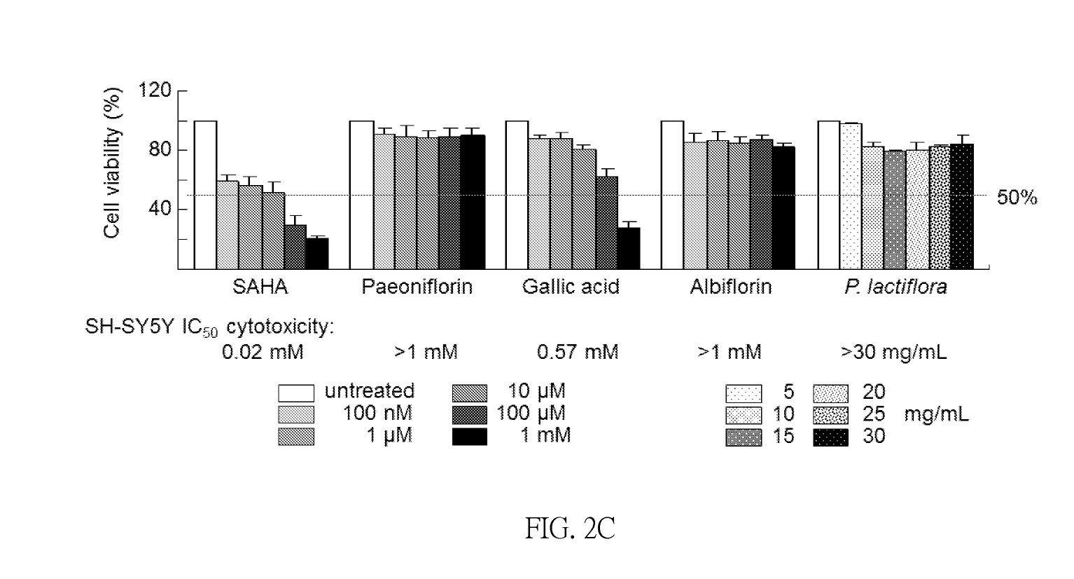

In the present example, the chemical profile of extract was analyzed and quantified by full-spectrum analytic HPLC. As shown in FIG. 2A, chromatographic patterns showed peaks at 230 nm corresponding to the retention time compatible with paeoniflorin, gallic acid and albiflorin. The amounts of paeoniflorin, gallic acid and albiflorin in extract of P. lactiflora were 2.27%, 0.30% and 0.73%, respectively, corresponding to 47.33 mM, 18.06 mM and 15.16 mM, respectively, in 1 g/mL extract. In MTT assays, the results of cytotoxicity, in which the treatment with the extract of P. lactiflora, paeoniflorin, gallic acid, albiflorin and SAHA against human embryonic kidney 293 and human neuroblastoma SH-SY5Y cells treated with for 24 hours, were shown in FIGS. 2B and 2C. The histone deacetylase inhibitor SAHA known to reduce SDS-insoluble polyQ aggregates was included for comparison. The IC₅₀ of the P. lactiflora extract, paeoniflorin and albiflori were calculated using the interpolation method. Both P. lactiflora extract and its constituents paeoniflorin and albiflorin had an IC₅₀ higher than the highest concentration tested (>30 mg/mL for P. lactiflora and >1 mM for paeoniflorin and albiflorin), suggesting their very low cytotoxicity.

P lactiflora Extract and Paeoniflorin Reduce ATXN3/Q₇₅ Aggregation on 293 Cell Model

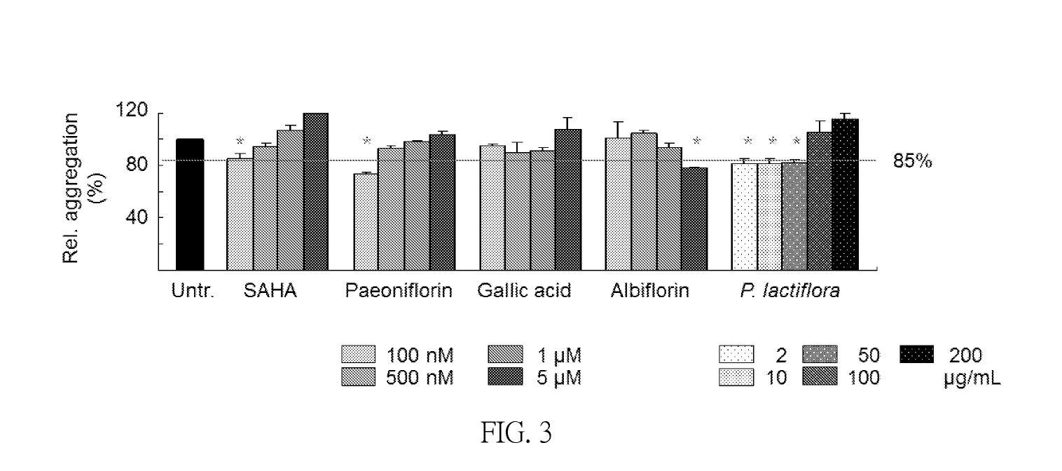

In the present example, the influences of the P. lactiflora extract and paeoniflorin in the ATXN3/Q₇₅-GFP cells were respectively examined. After 6 days of the treatment of doxycycline and oxaliplatin, the fluorescence microscopy images were observed, and aggregation percentage of ATXN3/Q₇₅-GFP cells untreated or treated with P. lactiflora (10 μg/mL), as well as paeoniflorin, gallic acid, albiflorin and HDAC inhibitor SAHA (100 nM) was assessed by high-content compound screen system. The result was shown in FIG. 3, in which SAHA served as a control of reducing the ATXN3/Q₇₅ aggregation. Referring to FIG. 3, HDAC inhibitor SAHA reduced the ATXN3/Q₇₅ aggregation to 85% (at 100 nM) as compared to untreated cells. While gallic acid did not display good aggregation-inhibitory potential (90˜95% at 100 nM˜1 μM), P. lactiflora (81˜82% at 2˜50 μg/mL), paeoniflorin (73% at 100 nM) and albiflorin (78% at 5 μM) had greater aggregation reduction potential than SAHA. The IC₅₀ cytotoxicity/effective (reduced the ATXN3/Q₇₅ aggregation to 85% or lower) dose ratio of SAHA, paeoniflorin, albiflorin and extract of P. lactiflora are 3800, >10000, >200 and >15000, respectively. Accordingly, paeoniflorin was regarded as a major active component for the aggregation inhibition in P. lactiflora.

P lactiflora Extract and Paeoniflorin Reduced ATXN3/Q₇₅ Aggregation on SH-SY5Y Cell Model

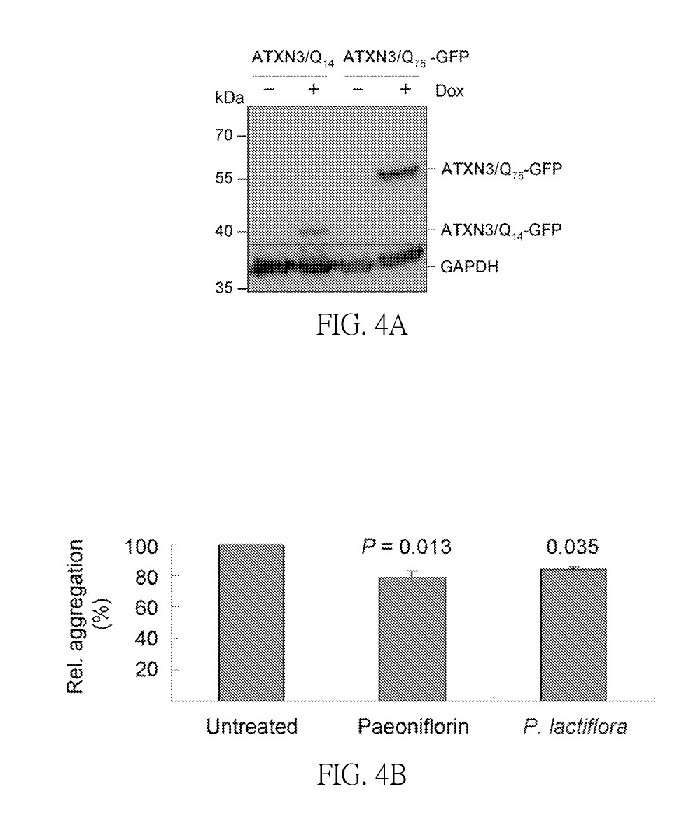

FIG. 4A shows Western blot analysis of protein expression in ATXN3/Q₇₅-GFP SH-SY5Y cells induced by doxycycline, wherein GFP-tagged 40˜57 kDa ATXN3/Q_{14˜75} protein in Dox-induced SH-SY5Y cells can be seen. Then, ATXN3/Q_{14˜75} SH-SY5Y cells were differentiated using trans-retinoic acid, and it can be found that the induced ATXN3/Q₇₅ formed aggregates in about 1% neuronal cells. Referring to FIG. 4B, the treatment of paeoniflorin or P. lactiflora led to 21% to 16% of aggregation reduction (P=0.013˜0.035) in ATXN3/Q₇₅ expressed neuronal cells.

Accordingly, these above results confirmed the aggregation-inhibitory effect of paeoniflorin and P. lactiflora in differentiated neurons. It is to be understood, however, that these examples are for illustrative purposes only and are not intended to limit the scope of the present invention.

In summary, the pharmaceutical composition provided by the present invention can efficiently inhibit spinocerebellar ataxia, give assistance to the treatment of spinocerebellar ataxia and slow down disease progression in Chinese herbal medicine therapy, thereby rendering better quality of life to patients.

Although the present invention has been explained in relation to its preferred embodiment, it is to be understood that many other possible modifications and variations can be made without departing from the spirit and scope of the invention as hereinafter claimed.

What is claimed is:

1. A method for inhibiting spinocerebellar ataxia, comprising:
administering an extract of Paeonia lactiflora to a subject in need,
wherein a concentration of the extract of Paeonia lactiflora is in the range from 1 μg/mL to 80 μg/mL.

2. The method of claim 1, wherein the concentration of the extract of Paeonia lactiflora is in the range from 1.5 μg/mL to 55 μg/mL.

3. The method of claim 1, wherein the extract of Paeonia lactiflora comprises at least one active component selected from a group consisting of paeoniflorin and albiflorin.

4. The method of claim 3, wherein the paeoniflorin has a concentration of 50 nM to 300 nM, and albiflorin has a concentration of 3 μM to 10 μM.

5. The method of claim 1, wherein the extract of Paeonia lactiflora further comprises: at least one of a pharmaceutically acceptable carrier, a diluent, or an excipient.

6. The method of claim 1, wherein the extract of Paeonia lactiflora is obtained by heating and extracting Paeonia lactiflora in water and filtering out a residue.

7. The method of claim 6, wherein the heating is performed to heat the water and Paeonia lactiflora to 90° C. to 100° C. for 30 to 60 minutes.

8. The method of claim 6, wherein the water is in an amount of 10 to 20 times of the weight of the Paeonia lactiflora.

9. A method for suppressing aggregation of polyglutamine, comprising:
administering an extract of Paeonia lactiflora to a subject in need,
wherein a concentration of the extract of Paeonia lactiflora is in the range from 1 μg/mL to 80 μg/mL.

10. The method of claim 9, wherein the extract of Paeonia lactiflora comprises at least one active component selected from a group consisting of paeoniflorin and albiflorin.


SEQUENCE LISTING (2 sequences)

SEQ ID NO: 1 - DNA, 28 bp, Artificial sequence
  Synthesized primer
attcagctaagtatgcaaggtagttcca

SEQ ID NO: 2 - DNA, 30 bp, Artificial Sequence
  Synthesized primer
catgccatggcatgtttttttccttctgtt